(19) United States
(12) Patent Application Publication
(10) Pub. No.: US 2017/0165386 A1
(43) Pub. Date: Jun. 15, 2017

(54) MOBILE ELECTRONIC DEVICE STORAGE AND CHARGING CABINET HAVING STERILIZATION FUNCTION

(71) Applicant: CHEN-SOURCE INC., Taoyuan City (TW)

(72) Inventor: Chieh-Ju HUANG, Taoyuan City (TW)

(21) Appl. No.: 14/966,842

(22) Filed: Dec. 11, 2015

(51) Int. Cl. A61L 2/10

(52) U.S. Cl. CPC A61L 2/10

(57) ABSTRACT

A mobile electronic device storage and charging cabinet includes a body defining therein an accommodation chamber and carriers with individual storage chambers mounted in the accommodation chamber for storing mobile electronic devices individually, a power supply module consisting of a control system and a connector module and mounted in the accommodation chamber with electrical connectors of the connector module respectively electrically connected to storage mobile electronic devices by cables, and a sterilizer consisting of a germicidal lamp set and power drive module and mounted in the body for killing microorganisms, molds, fungi and unicellular algae that are suspended in the air inside the body and adhered to the surfaces of storage mobile electronic devices with ultraviolet light from ultraviolet lamp tubes of the germicidal lamp set subject to the control of the power drive module, achieving optimal disinfection and sterilization effects and protecting the users from health risks.

### BACKGROUND OF THE INVENTION

### 1. Field of the Invention

The present invention relates to storage means, and more particularly to a mobile electronic device storage and charging cabinet having sterilization function, which has a sterilizer mounted in the body thereof controllable to drive an ultraviolet lamp to emit ultraviolet light onto the surface of storage electronic devices, providing optimal disinfection and sterilization effects, letting people operating the electronic devices less susceptible to disease transmission and reducing the risk of developing health problems.

### 2. Description of the Related Art

With fast development of electronic and multimedia technologies, advanced and sophisticated notebook computers, tablet computer, smart phone, PDA and many other mobile electronic devices have been continuously created and appeared on the market. These mobile electronic devices have the characteristics of small size, light weight and high mobility. With continuous creation of hardware and software functions, advanced mobile electronic devices provide high flexibility and applicability and are widely used in our daily life and daily work for entertainment and other applications. However, due to the characteristics of small size, light weight and high mobility, the internal electronic components of notebook computers, tablets and other advanced mobile electronic devices are of high precision design, and these mobile electronic devices can easily be damaged by impact. Many protective cases have been created for carrying and protecting notebook computers. However, these protective cases are commonly designed for holding one single notebook computer. Further, a school, organization or office may need to use a large number of notebook computers, tablet computers and/or other mobile electronic devices to satisfy different requirements for information sorting or different software applications. After each use, these mobile electronic devices may be directly left on desks. The use of a large amount of mobile electronic devices brings management inconvenience to the person in charge. Further, these mobile electronic devices can be easily stolen. Putting mobile electronic devices such as notebook computers or tablet computers in protective cases facilitates carrying and enhances the convenience of use. However, conventional protective cases for mobile electronic device do not provide a charging function, resulting in an inevitable problem in charging storage mobile electronic devices. Therefore, before bringing and using a mobile electronic device, the user needs to electrically connect the mobile electronic device to an external power source by a power adapter or battery charger, enabling the mobile electronic device to be fully charged. In consequently, the overall effect of use and convenience would be restricted.

Further, the use of tablet computers and other mobile electronic devices for educational applications has been gradually increased and widely spread. If a touch-operated tablet computer or other mobile electronic device is used by several persons, the surface of the touch-operated tablet computer or mobile electronic device can be contaminated with hands sweat, fingerprints, or even other dirt. Further, in homes, daycare centers, schools or medical institutes, bacteria, viruses, fungi and other pathogens can easily be spread via spit or direct contact, thus, touch-operated tablet computers and other mobile electronic devices for public use can become transmission media of contagious diseases, bringing health risks. Conventionally, it is the commonly to sterilize touch-operated tablet computers and other mobile electronic devices with chemical fungicide or alcohol. However, residual chemical fungicide or alcohol on the surface of a touch-operated tablet computer or other mobile electronic device can come in direct contact with the skin of the user, triggering skin reactions or causing toxic residues on the skin of the user. Further, after a long period of use, people may forget to sterilize the touch-operated tablet computer or mobile electronic device due to negligence or burnout, bringing health risks. Measures capable of solving the aforesaid problems are desired.

### SUMMARY OF THE INVENTION

The present invention has been accomplished under the circumstances in view. It is the main object of the present invention to provide a mobile electronic device storage and charging cabinet having sterilization function, which comprises a body defining therein an accommodation chamber and carriers with individual storage chambers mounted in the accommodation chamber for storing mobile electronic devices individually, a power supply module consisting of a control system and a connector module and mounted in the accommodation chamber with electrical connectors of the connector module respectively electrically connected to storage mobile electronic devices by cables for charging storage mobile electronic devices, and a sterilizer consisting of a germicidal lamp set and power drive module and mounted in the body for killing microorganisms, molds, fungi and unicellular algae that are suspended in the air inside the body and adhered to the surfaces of storage mobile electronic devices with ultraviolet light from ultraviolet lamp tubes of the germicidal lamp set subject to the control of the power drive module, achieving optimal disinfection and sterilization effects and protecting the users from health risks.

Preferably, the germicidal lamp set of the sterilizer further comprises a lamp shield affixed to the body horizontal frame plate above the lamp holder and over the at least one ultraviolet lamp tube to concentrate the emitted ultraviolet light toward storage mobile electronic devices, achieving optimal radiation effects. Using the germicidal lamp set to irradiate storage mobile electronic devices can effectively kill microorganisms, molds, fungi and unicellular algae that are suspended in the air inside the body of the mobile electronic device storage and charging cabinet and adhered to the surfaces of storage mobile electronic devices. Further, the germicidal lamp set has the advantages of long lifespan, low maintenance cost, low unit cost of electricity, and high disinfection and sterilization effects without using any chemical fungicide or alcohol. Further, disinfection and sterilization can be implemented only after turning on the germicidal lamp set, preventing from causing inhalation injuries. After the germicidal lamp set is turned off, no ultraviolet light will be left. Further, the arrangement of the lamp shield prohibits the emitted ultraviolet light from directly radiating into human body, enhancing practicality and applicability.

Preferably, the body further comprises two sets of upright support plates vertically disposed at two opposite lateral sides relative to said accommodation chamber, two door panel receiving spaces respectively defined between two opposite lateral sides of said accommodation chamber and said two sets of upright support plates, two sets of rails respectively horizontally mounted in said door panel receiving spaces and affixed to the respective said upright support plates at different elevations, and two door panels respectively coupled to said rails and movable between a closed position to close said accommodation chamber and an open position to open said accommodation chamber.

Preferably, the body of the mobile electronic device storage and charging cabinet further comprises two door panels for closing the accommodation chamber; the power drive module of the sterilizer further comprises a normally open tact switch, which switches off the germicidal lamp set when the door panels are opened, or switches on the germicidal lamp set when the door panels are closed, assuring a high level of safety and avoiding hazards caused by radiation of the germicidal lamp set due to that the door panels are not properly closed.

Preferably, the power supply module further comprises an expansion connector module. The expansion connector module comprises a firmware update jack electrically connected to the power drive module by a cable. Thus, the user can use the firmware update jack to update the firmware of the power drive module to the latest edition, improving the performance and reliability of the power drive module.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is an oblique top elevational view of a mobile electronic device storage and charging cabinet in accordance with the present invention.

FIG. 2 is an exploded view of the mobile electronic device storage and charging cabinet in accordance with the present invention.

FIG. 3 corresponds to FIG. 2 when viewed from another angle.

FIG. 4 is a partial exploded view of the body and the sterilizer according to the present invention.

FIG. 5 is a schematic drawing of the invention, illustrating the mobile electronic device storage and charging cabinet opened before insertion of mobile electronic devices.

FIG. 6 is a schematic front view of the present invention, illustrating a charging operation of the power supply module of the mobile electronic device storage and charging cabinet.

FIG. 7 is a schematic front view of the present invention, illustrating a sterilization operation of the sterilizer of mobile electronic device storage and charging cabinet.

FIG. 8 is a schematic side view of the present invention, illustrating a sterilization operation of the sterilizer of mobile electronic device storage and charging cabinet.

### DETAILED DESCRIPTION OF THE PREFERRED EMBODIMENT

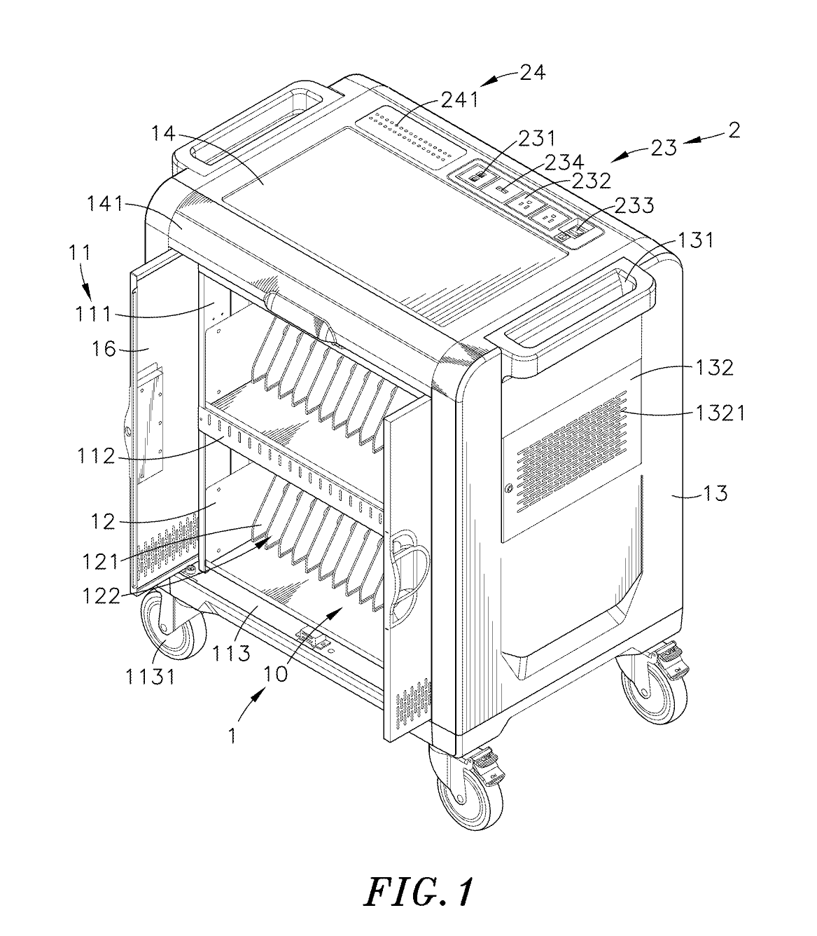

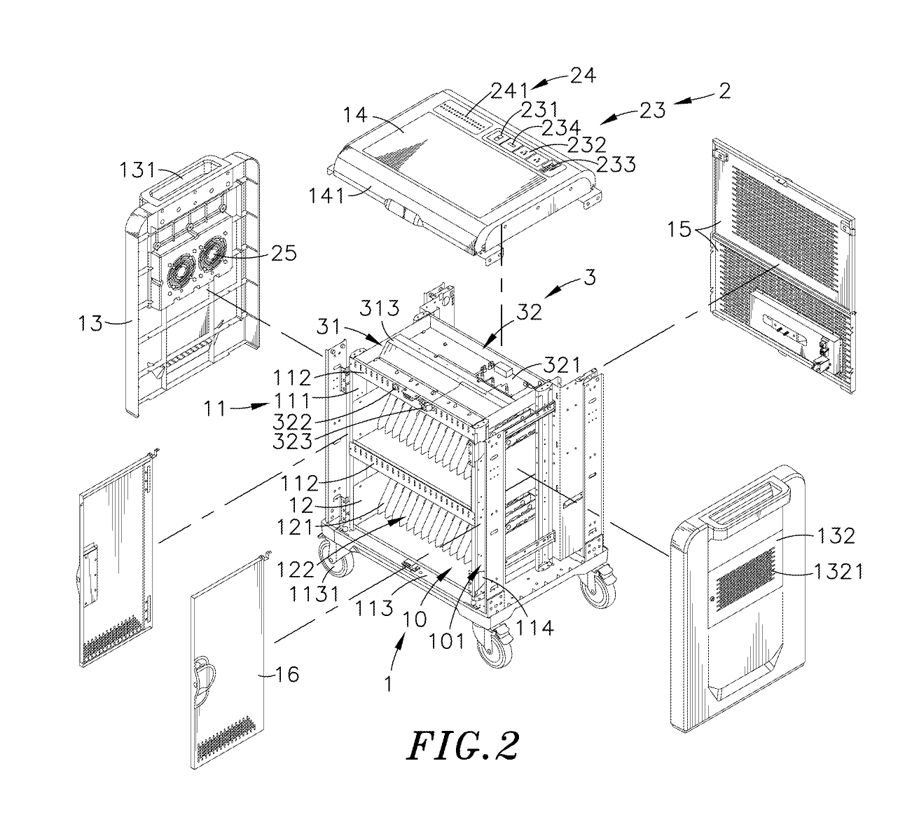

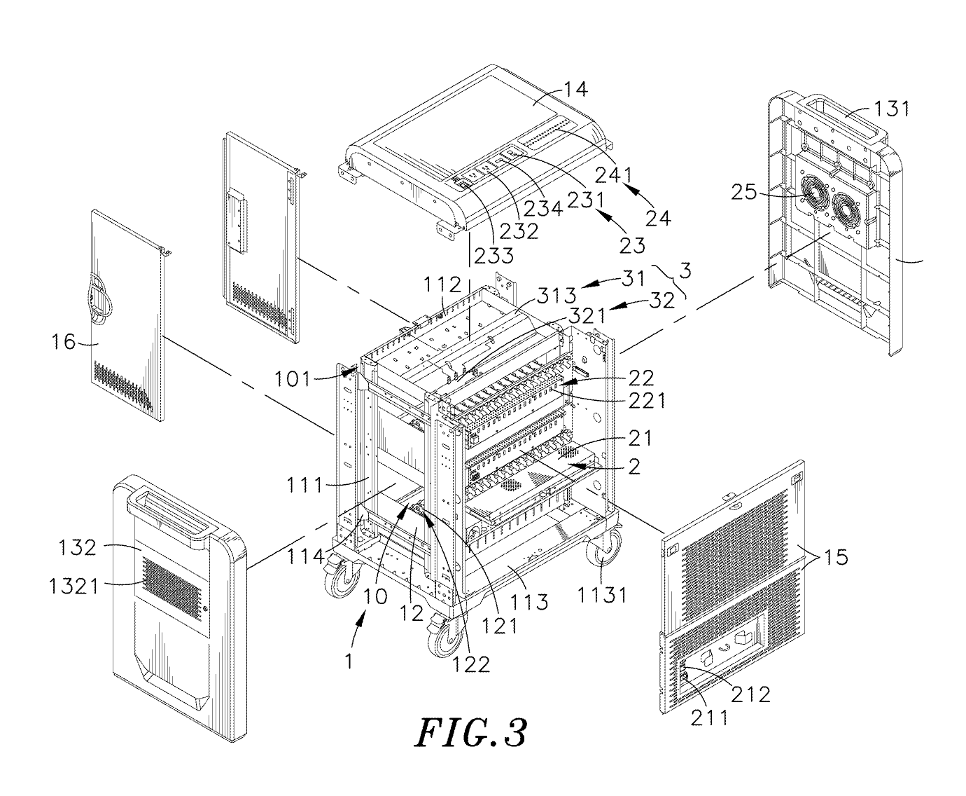

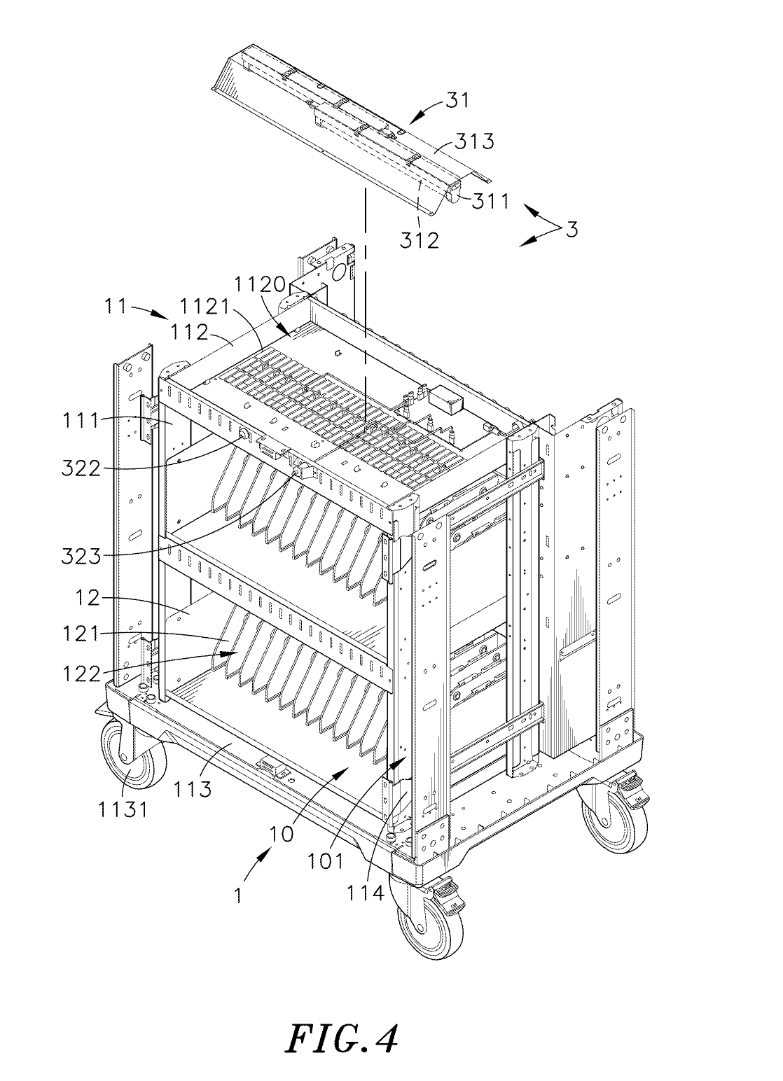

Referring to FIGS. 1-4, a mobile electronic device storage and charging cabinet having sterilization function in accordance with the present invention is shown. The mobile electronic device storage and charging cabinet comprises a body 1, and a power supply module 2.

The body 1 comprises a framework 11. The framework 11 comprises an accommodation chamber 10, two sets of upright support plates 111 vertically and symmetrically disposed at two opposite lateral sides relative to the accommodation chamber 10, two door panel receiving spaces 101 respectively defined between two opposite lateral sides of the accommodation chamber 10 and the two sets of upright support plates 111, a plurality of, for example, two horizontal frame plates 112 connected between the upright support plates 111 at different elevations and dividing the accommodation chamber 10 into upper and lower chambers, a mounting chamber 1120 defined in the upper horizontal frame plate 112, a plurality of slots 1121 located in a bottom side of the mounting chamber 1120 in communication with the inside space of the accommodation chamber 10, a bottom panel 113 horizontally located at a bottom side thereof the framework 11, and a plurality of caster 1131 pivotally mounted in four corners of a bottom wall of the bottom panel 113.

The body 1 comprises a plurality of, for example, two carriers 12 respectively mounted on the lower horizontal frame plate 112 and bottom panel 113 of the framework 11, each carrier 12 comprising a plurality of spacer plates 121 and a plurality of individual storage chambers 122 respectively defined between each two adjacent spacer plates 121, two upright side panels 13 covered on two opposite lateral sides of the framework 11, each upright side panel 13 comprising a grip 131 located at a top side thereof and an outer cover 132 with air vents 1321, a top panel 14 covered on a top side of the framework 11 and carrying therein a detachable tray 141, a shielding panel 15 covered on a back side of the framework 11, two sets of rails 114 respectively horizontally mounted in the door panel receiving spaces 101 of the framework 11 and affixed to the respective upright support plates 111 at different elevations, and two door panels 16 respectively coupled to the rails 114. Normally, the two door panels 16 are kept on one same plane in a closed position at a front side of the framework 11 to close the mobile electronic device storage and charging cabinet. The user can turn the two door panels 16 outwardly from the closed position through 90° angle relative to the framework 11 and then push the two door panels 16 backwardly along the respective rails 114 into the inside of the door panel receiving spaces 101 of the framework 11 in an open position to open the mobile electronic device storage and charging cabinet, or, pull the two door panels 16 along the respective rails 114 out of the respective door panel receiving spaces 101 and then turn the two door panels 16 inwardly through 90° angle to the closed position again.

The power supply module 2 comprises a control system 21 and at least one connector module 22 electrically connected to the control system 21. The control system 21 comprises a control circuit (not shown) for processing electronic signals, a power adapter 211 electrically connected to the control circuit, and a power switch 212 for switching on/off the power adapter 211. The power adapter 211 can be a socket type power adapter or a plug type power adapter. In the case that the power adapter 211 is a socket type power adapter, connect an electric plug at one end of a power cable to the power adapter 211 and then connect another electric plug at an opposite end of the power cable to an electrical outlet of an external power source (such as city power supply, power supply device, power generator, etc.) for enabling the control system 21 to convert inputted AC power supply into a regulated DC power supply for the connector module 22. The connector module 22 comprises a plurality of electrical connectors 221 respectively electrically connected to the control system 21 by cables. Further, the electrical connectors 221 can be selected from the group of USB2.0 Type-A female, USB3.0 Type-A female, etc.

The power supply module 2 further comprises an expansion connector module 23 and an indicator light module 24 electrically connected to the connector module 22 and the control system 21 by cables (not shown). The expansion connector module 23 comprises a signal connector 231, a plurality of power sockets 232, and an on/off switch 233. The signal connector 231 can be a USB female connector, a network connector (RJ45), an electrical audio connector, or any other electrical connector for signal input/output. Further, the indicator light module 24 comprises a plurality of light-emitting diodes 241 for the indication of the operating status (such as charging, signal transmission, etc.) of the respective electrical connectors 221 of the connector module 22. The power supply module 2 further comprises a plurality of cooling fans 25 electrically connected to the control system 21 by cables (not shown) and controllable to draw outside cold air toward the inside of the body 1 to lower the internal temperature.

The sterilizer 3 comprises a germicidal lamp set 31 and a power drive module 32 electrically connected to the germicidal lamp set 31. The germicidal lamp set 31 comprises a lamp holder 311, a plurality of ultraviolet lamp tubes 312 mounted in the lamp holder 311, and a lamp shield 313 affixed to the lamp holder 311 over the ultraviolet lamp tube 312. Further, the ultraviolet lamp tubes 312 emit the short wavelength ultraviolet radiation (UV-C, λ=100˜280 nm). The power drive module 32 comprises a circuit board 321 electrically connected to the lamp holder 311 of the germicidal lamp set 31 by a cable (not shown). The circuit board 321 has installed therein a rectifier or starter (not shown) for providing stabilized power supply, and a push button switch 322 and a normally open tact switch 323 for power control. Further, the circuit board 321 can be electrically connected to the control system 21 of the power supply module 2 so that the desired power supply can be provided to the power drive module 32 through the control system 21. Further, in actual application, the germicidal lamp set 31 and power drive module 32 of the sterilizer 3 can be configured according to different specifications and types to satisfy different requirements. Further, the germicidal lamp set 31 and the power drive module 32 can be separately arranged or integrated in one single unit subject to actual application requirement or customized demand. Further, the lamp holder 311 can be variously embodied to mate with different sizes or designs of ultraviolet lamp tubes 312 without departing from the spirit and scope of the invention.

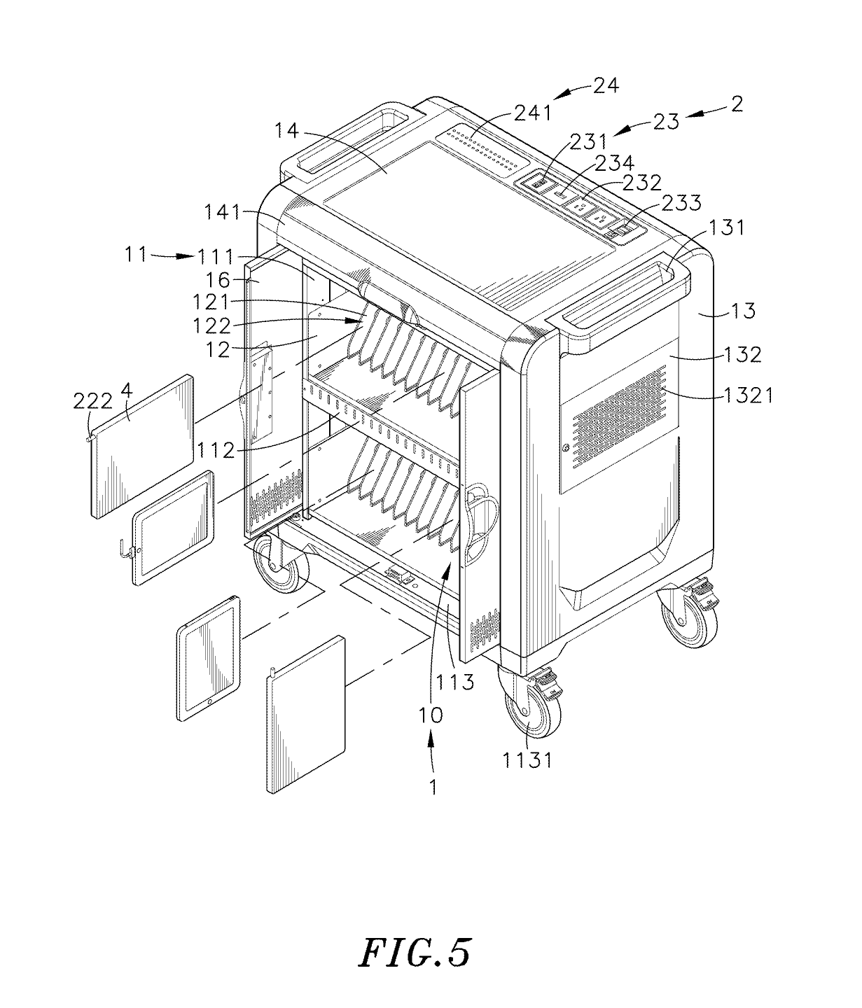

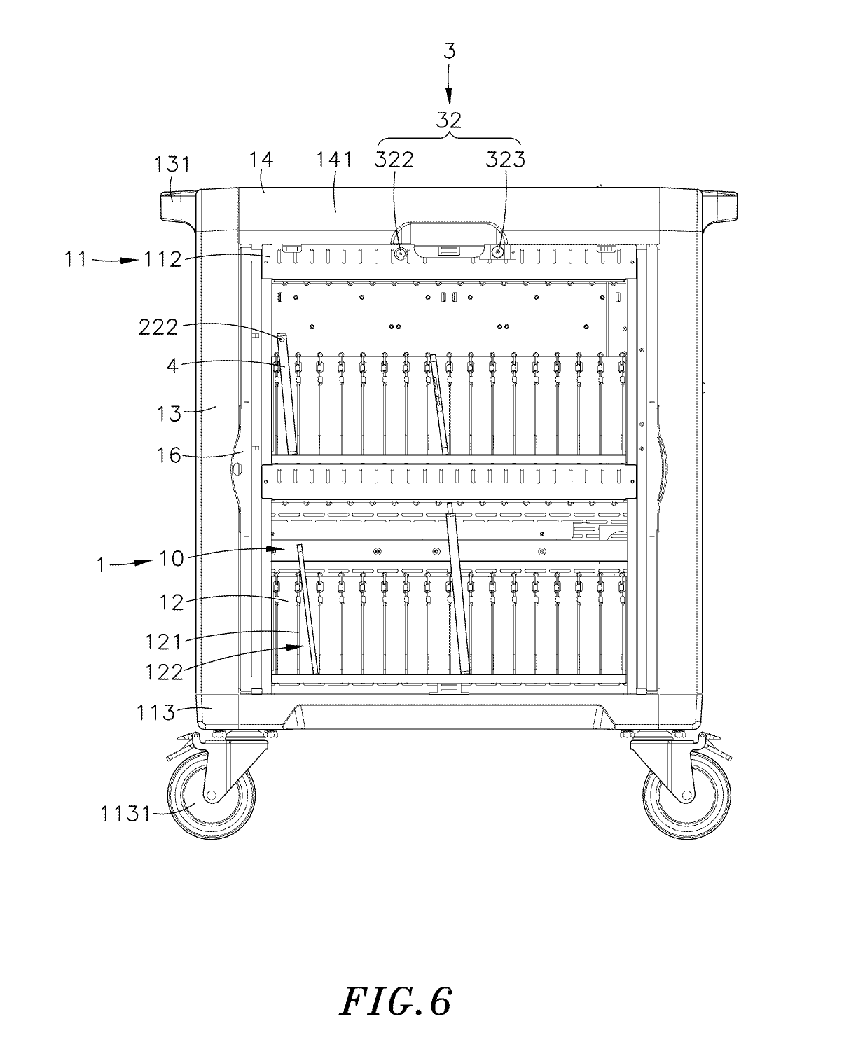

In installation, mount the control system 21 of the power supply module 2 in the accommodation chamber 10 of the body 1 at a back side relative to the carriers 12 to let the power adapter 211 and the power switch 212 be exposed to the outside of the shielding panel 15, and then mount the connector module 22 between the two sets of upright support plates 111 at the rear side of the framework 11, and then connect the electrical plugs at the respective one ends of the cables 222 (see FIGS. 5 and 6) to the electrical connectors 221 and then insert the electrical plugs at respective other ends of the cables 222 through the carriers 12 for enabling the cables 222 to be secured in place by cable clamps (not shown) at the bottom side of the spacer plates 121, facilitating user arranging and connecting cables.

Thereafter, mount the expansion connector module 23 and indicator light module 24 of the power supply module 2 in the top panel 14 of the body 1, letting the signal connector 231, the power sockets 232, the on/off switch 233 and the light-emitting diodes 241 be exposed to the outside of the top panel 14, and then mount the cooling fans 25 at the upright side panels 13 at an inner side to corresponding to the air vent 1321 in the outer cover 132, and then mount the germicidal lamp set 31 and power drive module 32 of the sterilizer 3 in the mounting chamber 1120 within the upper horizontal frame plate 112 of the framework 11, and then affix the lamp shield 313 of the germicidal lamp set 31 to the upper horizontal frame plate 112 to keep the lamp holder 311 and the ultraviolet lamp tubes 312 in the lamp holder 311 be disposed above the carriers 12, and then set the push button switch 322 and normally open tact switch 323 of the power drive module 32 in the front side of the upper horizontal frame plate 112 to expose to the front open side of the accommodation chamber 10 of the body 1, and thus, the installation of the mobile electronic device storage and charging cabinet is done.

Further, the aforesaid expansion connector module 23 further comprises a firmware update jack 234 (for example, USB connector) electrically connected to the power drive module 32 by a cable. Thus, the user can use the firmware update jack 234 to update the firmware of the power drive module 32 to the latest edition, improving the performance and reliability of the power drive module 32, for example, allowing the power drive module 32 to control the operating time of the germicidal lamp set 31, i.e., to automatically turn off the germicidal lamp set 31 when the set time is up.

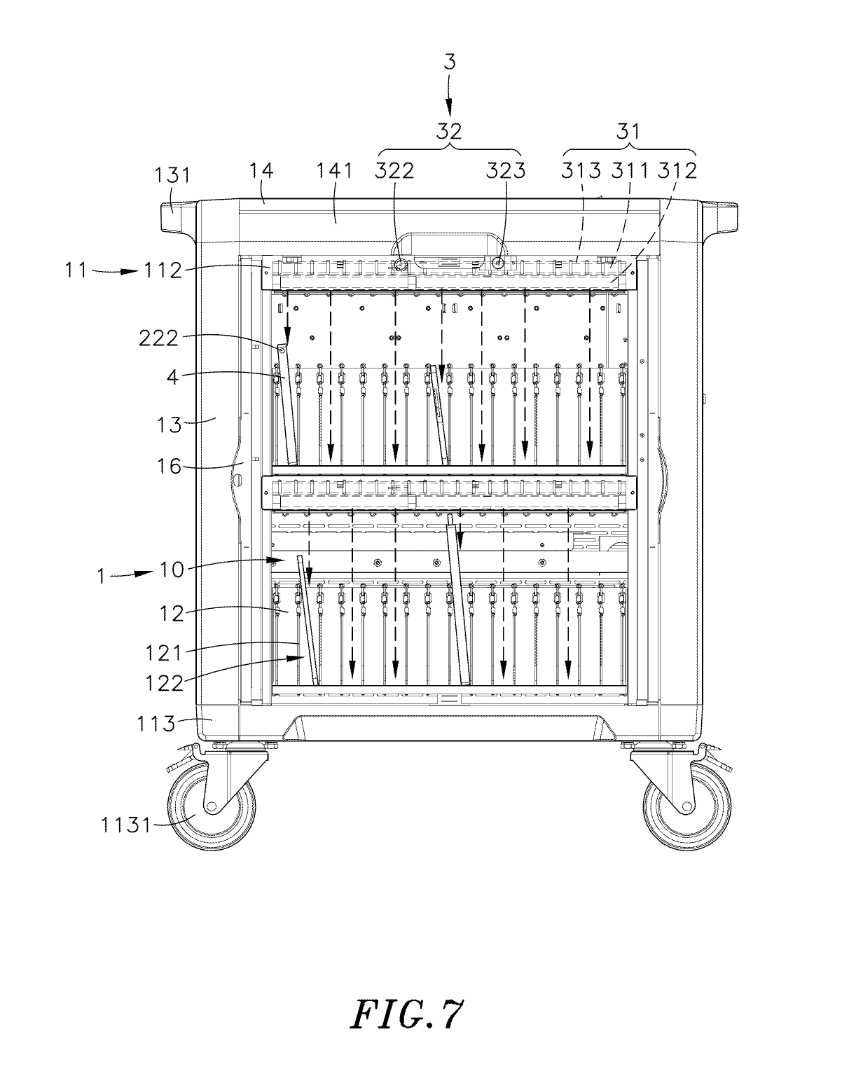

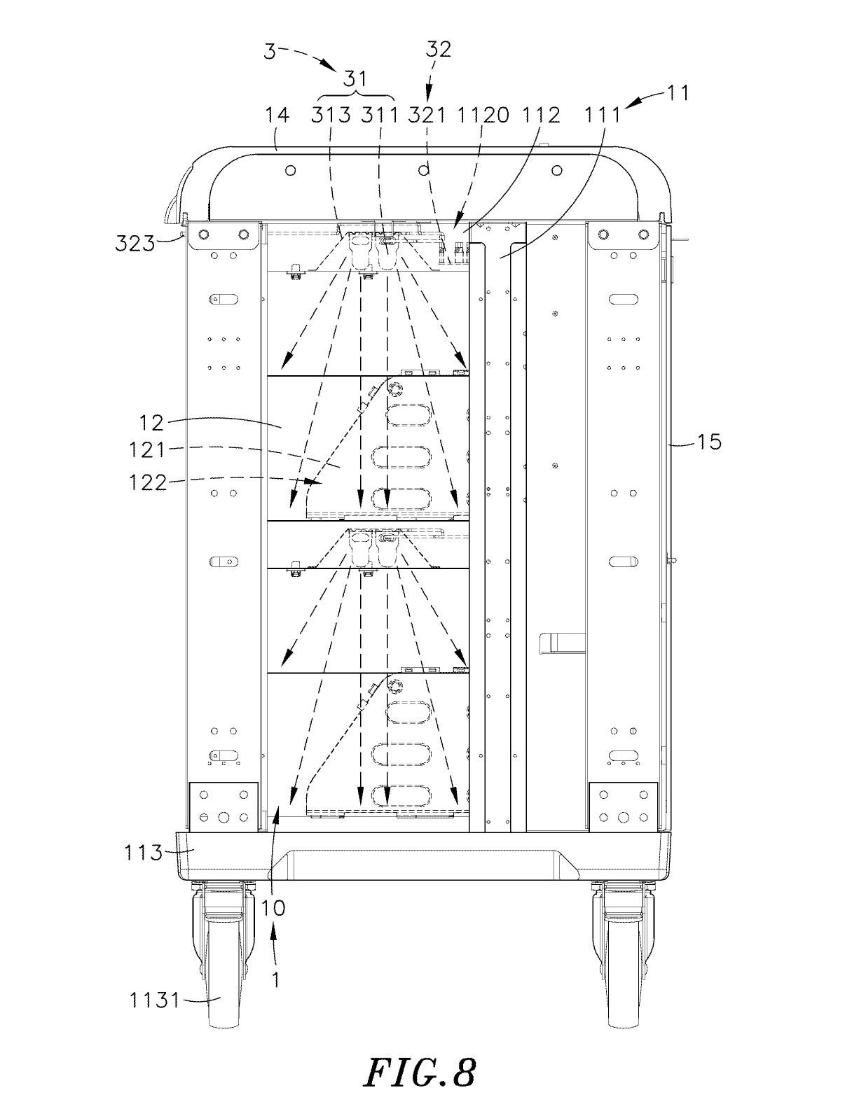

Referring to FIGS. 5-8, an exploded view of the mobile electronic device storage and charging cabinet before storage of mobile electronic devices, a front view of the mobile electronic device storage and charging cabinet during a charging operation, a partial schematic front view of the mobile electronic device storage and charging cabinet during a sterilization operation and a partial schematic side view of the mobile electronic device storage and charging cabinet are shown. As illustrated, when using the mobile electronic device storage and charging cabinet, the user can connect the electrical plugs (not shown) at the respective other ends of the cables 222 of the connector module 22 to the respective mating electrical sockets (not shown) of respective mobile electronic devices 4 (such as notebook computers, tablet computers, etc.) to electrically connect the mobile electronic devices 4 to the connector module 22, and then vertically set the mobile electronic devices 4 in respective individual storage chambers 122 in the carriers 12 for easy access and management. After positioning of the mobile electronic devices 4 in the respective individual storage chambers 122, operate the control system 21 of the power supply module 2 to provide power supply to the connector module 22, enabling rectified stabilized power supply to be transmitted through the cables 222 to charge the mobile electronic devices 4 and signals to be transmitted through the signal connector 231 of the expansion connector module 23 the mobile electronic devices 4 for updating records or software programs conveniently without any extra tools, saving much labor and time, improving mobile electronic device charging efficiency and rendering a convenience effect on the overall use. Further, after the door panels 16 of the body 1 are closed, the door panels 16 can be locked in the closed position, preventing evil persons from stealing or damaging storage mobile electronic devices 4 and offering a high level of anti-theft security.

When the user presses on the push button switch 322 of the power drive module 32 of the sterilizer 3, the user can close the door panels 16 over the accommodation chamber 10 of the framework 11 of the body 1 to switch the normally open tact switch 323 of the power drive module 32 from the normally open status to the close status, enabling the power drive module 32 to drive on the germicidal lamp set 31, and thus, this security arrangement assures a high level of safety, avoiding hazards caused by radiation of the germicidal lamp set 31 as the door panels 16 are not properly closed. When the power drive module 32 is started to turn on the ultraviolet lamp tubes 312 in the lamp holder 311 of the germicidal lamp set 31, the ultraviolet lamp tubes 312 are turned on to emit ultraviolet light through the slots 1121 in the horizontal frame plate 112 toward storage mobile electronic devices 4 in the individual storage chambers 122 in the carriers 12. Further, a lamp shield 313 can be a reflector for reflecting emitted ultraviolet light from the ultraviolet lamp tubes 312 in direction toward the slots 1121 and storage mobile electronic devices 4 to kill microorganisms, molds, fungi and unicellular algae that are suspended in the air inside the body 1 and adhered to the surfaces of storage mobile electronic devices 4. Further, the installation locations and angles of the ultraviolet lamp tubes 312 and the lamp holder 311 are precisely calculated so that emitted ultraviolet light can be evenly distributed over the surface of every storage mobile electronic device 4, achieving optimal disinfection and sterilization effects and protecting the users susceptible to disease transmission (such as young children, the elderly, patients and those with poor immunity) from health risks. Further, the germicidal lamp set 31 has the advantages of long lifespan, low maintenance cost, low unit cost of electricity, and high disinfection and sterilization effects without using any chemical fungicide or alcohol. Further, disinfection and sterilization can be implemented only after turning on the germicidal lamp set 31, preventing from causing inhalation injuries. After the germicidal lamp set 31 is turned off, no ultraviolet light will be left. Further, the arrangement of the lamp shield 313 prohibits the emitted ultraviolet light from directly radiating into human body, enhancing practicality and applicability.

In conclusion, the invention provides a mobile electronic device storage and charging cabinet having sterilization function, which comprises a body 1 having an accommodation chamber 10 defined in a framework 11 thereof, carriers 12 mounted in the accommodation chamber 10 for holding mobile electronic devices 4, and a sterilizer 3 consisting of a germicidal lamp set 31 and power drive module 32 and mounted in the top side of the body 1 for sterilizing storage mobile electronic devices 4 with ultraviolet light from ultraviolet lamp tubes 312 in a lamp holder 311 of the germicidal lamp set 31 subject to the control of the power drive module 32 so to kill microorganisms, molds, fungi and unicellular algae that are suspended in the air inside the body 1 or adhered to the surfaces of storage mobile electronic devices 4, achieving optimal disinfection and sterilization effects and protecting the users from health risks.

Although a particular embodiment of the invention has been described in detail for purposes of illustration, various modifications and enhancements may be made without departing from the spirit and scope of the invention. Accordingly, the invention is not to be limited except as by the appended claims.

What the invention claimed is:

1. A mobile electronic device storage and charging cabinet, comprising:
a body comprising a framework defining therein an accommodation chamber, and at least one carrier mounted in said accommodation chamber, each said carrier comprising a plurality of spacer plates and a plurality of individual storage chambers respectively defined between each two adjacent said spacer plates for holding mobile electronic devices individually;
a power supply module mounted in said accommodation chamber of said body, said power supply module comprising a control system for connection to an external power source for power input and a connector module electrically connected to said control system, said connector module comprising a plurality of mating electrical connectors and a plurality of cables for electrically connecting said mating electrical connectors to the mobile electronic devices being stored in said individual storage chambers to charge the stored said mobile electronic devices; and
a sterilizer mounted in said accommodation chamber of said body for sterilizing the stored said mobile electronic devices with ultraviolet light, said sterilizer comprising a germicidal lamp set disposed above said at least one carrier and a power drive module electrically coupled with said germicidal lamp set, said germicidal lamp set comprising a lamp holder and at least one ultraviolet lamp tube mounted in said lamp holder and drivable by said power drive module to emit ultraviolet light toward the stored said mobile electronic devices in said at least one carrier.

2. The mobile electronic device storage and charging cabinet as claimed in claim 1, wherein said body further comprises two sets of upright support plates vertically disposed at two opposite lateral sides relative to said accommodation chamber, two door panel receiving spaces respectively defined between two opposite lateral sides of said accommodation chamber and said two sets of upright support plates, two sets of rails respectively horizontally mounted in said door panel receiving spaces and affixed to the respective said upright support plates at different elevations, and two door panels respectively coupled to said rails and movable between a closed position to close said accommodation chamber and an open position to open said accommodation chamber.

3. The mobile electronic device storage and charging cabinet as claimed in claim 2, wherein said power drive module of said sterilizer comprises a circuit board, and a normally open tact switch mounted at said circuit board and normally disposed in an open position to switch off said germicidal lamp set and switchable by said door panels from said open position to a close position to switch on said germicidal lamp set when said door panels are in said closed position.

4. The mobile electronic device storage and charging cabinet as claimed in claim 1, wherein said body further comprises a plurality of horizontal frame plates transversely mounted in said framework at different elevations, a mounting chamber defined in the topmost said horizontal frame plate, and a plurality of slots cut through a bottom wall of said mounting chamber for the passing of the emitted ultraviolet light from said germicidal lamp set toward the stored said mobile electronic devices in said at least one carrier.

5. The mobile electronic device storage and charging cabinet as claimed in claim 4, wherein said germicidal lamp set of said sterilizer further comprises a lamp shield spaced above said lamp holder over said at least one ultraviolet lamp tube and affixed to the topmost said horizontal frame plate.

6. The mobile electronic device storage and charging cabinet as claimed in claim 4, wherein said power drive module of said sterilizer comprises a circuit board, and a push button switch and said normally open tact switch installed in said circuit board and exposed to the outside of the topmost said horizontal frame plate.

7. The mobile electronic device storage and charging cabinet as claimed in claim 1, wherein said body further comprises two upright side panels respectively covered on two opposite lateral sides of said framework, each said upright side panel comprising a grip located at a top side thereof and an outer cover located at an outer side thereof, said outer cover comprising a plurality of air vents; said power supply module further comprises a plurality of cooling fans respectively mounted at said upright side panels corresponding to the respective said air vents and respectively electrically connected to said control system.

8. The mobile electronic device storage and charging cabinet as claimed in claim 1, wherein said body further comprises a top panel covered on said framework at a top side, a detachable tray removably received in said top panel, and a shielding panel covered on a back side of said framework.

9. The mobile electronic device storage and charging cabinet as claimed in claim 1, wherein said power supply module further comprises an expansion connector module electrically connected to said control system and said connector module, said expansion connector module comprising a signal connector, a plurality of power sockets and an on/off switch.

10. The mobile electronic device storage and charging cabinet as claimed in claim 9, wherein said expansion connector module further comprises a firmware update jack electrically connected to said power drive module for enabling a user to update the firmware of said power drive module for time control.

11. The mobile electronic device storage and charging cabinet as claimed in claim 1, wherein said power supply module further comprises an indicator light module electrically connected to said control system and said connector module and adapted for indicating the operating status of the respective said electrical connectors of said connector module.

12. The mobile electronic device storage and charging cabinet as claimed in claim 1, wherein said at least one ultraviolet lamp tube of said sterilizer germicidal lamp set is configured to emit the short wavelength ultraviolet radiation (UV-C, λ=100˜280 nm).
